(19) United States
(12) Patent Application Publication
(10) Pub. No.: US 2021/0401941 A1
(43) Pub. Date: Dec. 30, 2021

(54) MULTILAYER DRUG DELIVERY COATING FOR CONTACT LENS

(71) Applicant: UNIVERSITY OF PITTSBURGH - OF THE COMMONWEALTH SYSTEM OF HIGHER EDUCAITON, Pittsburgh, PA (US)

(72) Inventors: Mangesh Kulkarni, Sewickley, PA (US); Alexis Nolfi, Moon Township, PA (US); Bryan Nicklaus Brown, Pittsburgh, PA (US); Vishal Jhanji, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 17/469,032

(22) Filed: Sep. 8, 2021

Related U.S. Application Data
(63) Continuation of application No. PCT/US2020/021725, filed on Mar. 9, 2020.
(60) Provisional application No. 62/815,888, filed on Mar. 8, 2019.

(51) Int. Cl. A61K 38/20; A61K 31/737; A61P 27/04; G02B 1/04; G02B 1/08; G02B 1/10

(52) U.S. Cl. CPC A61K 38/2026; A61K 31/737; G02B 1/10; G02B 1/043; G02B 1/08; A61P 27/04

(57) ABSTRACT

Ophthalmic devices coated with an active agent eluting coating are provided herein. Placement of the coated ophthalmic devices on the surface of eye results in modulation of cells responding to an immune modifying agent and reducing inflammation-related complications in the eye. Methods for treating ocular disorders are also provided herein. The disclosed subject matter is based, in part, on the discovery that ophthalmic devices coated with a cytokine eluting coating can shift early-stage macrophage polarization associated with alleviation of symptoms and causes of inflammatory ocular disorders.

### 1. CROSS-REFERENCE TO RELATED APPLICATIONS

This application is a continuation of International Patent Application No. PCT/US2020/021725, filed Mar. 9, 2020, which claims priority to U.S. Provisional Application No. 62/815,888, filed Mar. 8, 2019, the contents of which are hereby incorporated by reference in their entireties.

### 2. FIELD

The presently disclosed subject matter relates to ophthalmic devices coated with an active agent eluting coating, wherein implantation of the coated ophthalmic devices results in modulation of a whole class of cells that can respond to an immune modifying agent with reduced inflammation-related complications in eyes.

### 3. BACKGROUND OF THE INVENTION

Certain eye disorders are characterized by aspects such as dryness and irritation of the eye. Such eye disorders are multifaceted disorders with implications for both tear quality and integrity of the ocular surface. Depending on the population studied and diagnosis criteria, the prevalence of such eye disease has been reported to occur in up to 33.7% of the population. Currently, treatment options are limited and focus on either temporary symptomatic relief (e.g., the frequent instillation of artificial tears) or the targeting of limited aspects of the immune system. This includes topical treatment with cyclosporine, of which there are only two FDA approved commercially available formulations (Restasis®, Cequa™), which makes the drug very expensive and sometimes inaccessible to patients. Steroid eye drops are also occasionally used; however, the side effects (e.g., cataract development, glaucoma, etc.) can be debilitating. Oral treatments to treat dry eye, such as doxycycline, have their own concerns, coupled with the obvious limitation of using a systemic drug to treat a localized problem. With all of these treatments, patient compliance is a major issue, especially when necessitating the need to apply potentially costly eye drops numerous times a day.

Accordingly, there is a need in the art for new techniques that provide effective treatments with optimal dosage for eye diseases.

### 4. SUMMARY OF THE INVENTION

The presently disclosed subject matter relates to ophthalmic devices uniformly coated with an active agent eluting coating, wherein implantation of the coated ophthalmic devices results in modulation of a local immune reaction and reduced inflammation-related complications in eyes. The presently disclosed subject matter also relates to methods for treating ocular disorders. The disclosed subject matter is based, at least in part, on the discovery that ophthalmic devices coated with a cytokine eluting coating resulted in the shift of early-stage macrophage polarization that was associated with alleviation of symptoms and causes of inflammatory ocular disorders compared to uncoated biomaterials.

The presently disclosed subject matter provides a contact lens for treating ocular disorders comprising a lens body and a uniform coating on the lens body, wherein the coating comprises a plurality of polycation layers and a plurality of polyanion layers, and wherein at least one layer of the coating includes an M2 polarizing active agent. In certain embodiments, the plurality of polycation layers and the plurality of polyanion layers alternate to form a plurality of bilayers.

In certain embodiments, the lens body can be a silicone hydrogel lens body. In certain embodiments, the contact lenses can be selected from the group consisting of balafilcon A, lotrafilcon A, lotrafilcon B, etafilcon A, narafilcon A, galyfilcon A, senofilcon A, ocufilcon D, hioxifilicon A, enfilcon A, comfilcon A, nesofilcon A, filicon II 3, deleficon A, methafilcon A, methafilcon B, vifilcon A, phemfilcon A, nelfilcon A, stenfilcon A, polymacon, hefilcon B, tetrafilcon A, omafilcon A, polymacon B, hilafilcon B, alphafilcon A, and combinations thereof.

In certain embodiments, the ocular disorder is selected from the group consisting of allergic, bacterial, chemical or viral conjunctivitis, blepharitis, dry eye syndrome, sub-conjunctival hematomas, corneal abrasion, uveitis, and combinations thereof.

In certain embodiments, the polycation in at least one polycation layer is selected from the group consisting of a polysaccharide, a protein, a synthetic polypeptide, a synthetic polyamine, a synthetic polymer, a positively charged polymer or copolymer, and combinations thereof.

In certain embodiments, the polyanion in at least one polyanion layer is selected from the group consisting of a polysaccharide, a protein, a synthetic polypeptide, a synthetic polyamine, a synthetic polymer, and combinations thereof.

In certain embodiments, the polycation is chitosan and the polyanion is dermatan sulfate. In certain embodiments, the M2 polarizing active agent is selected from the group consisting of IL-4, IL-10, IL-13, TGF-β, HGF, and combinations thereof.

In certain embodiments, a thickness of the coating is from about 0.5 nm to about 500 μm.

In certain embodiments, the at least one layer of the coating comprises dermatan sulfate and the M2 polarizing active agent. In certain embodiments, the M2 polarizing active agent and the dermatan sulfate are present in a ratio between about 1:10 to about 1:2000.

In certain embodiments, the coating comprises a macrophage-related enzyme. In certain embodiments, the macrophage-related enzyme adjusts a release rate of the active agent from the coating.

In certain embodiments, the coating is placed on the lens body without altering an optical property of the contact lens, wherein the optical property includes vision correction. In certain embodiments, the coating is degraded without altering an optical property of the contact lens, wherein the optical property includes vision correction. In certain embodiments, the contact lens simultaneously corrects vision of a subject during the release of active agents from the coating.

In certain embodiments, the coating is uniformly coated on a surface of the lens body without being exposed to plasma gas.

The presently disclosed subject matter also provides for a method for treating ocular disorders comprising: placing a contact lens on a surface of an eye, wherein the contact lens comprises (a) a lens body; and (b) a uniform coating thereon, wherein the coating comprises a plurality of polycation layers and a plurality of polyanion layers, and wherein at least one layer of the coating comprises an M2 polarizing active agent.

In certain embodiments, the method further comprises alleviating at least one symptom of the ocular disorder, wherein the at least one symptom is selected from the group consisting of redness, itching, burning, foreign body sensation, watery eyes, dry eyes, swelling, pain, clouding of vision, secretion of pus, sticking eyelids, altered sensitivity to light, and a combination of thereof.

In certain embodiments, the method further comprises sterilizing the contact lens without altering an architecture or a topography of the coating.

In certain embodiments of the method, the contact lens is worn continuously for about 30 days. In certain embodiments, the method further comprises delivering a supplement solution to the eye. In certain embodiments, the supplement solution comprises artificial tears. In certain embodiments, the supplement solution comprises a macrophage-related enzyme or protein.

In certain embodiments of the method, the coating is placed on the lens body without altering an optical property of the contact lens, wherein the optical property includes vision correction. In certain embodiments, the coating is degraded without altering an optical property of the contact lens, wherein the optical property includes vision correction. In certain embodiments, the contact lens simultaneously corrects vision of a subject during the release of active agents from the coating. In certain embodiments of the method, the coating is uniformly coated on a surface of the lens body without being exposed to plasma gas.

In non-limiting embodiments, the ocular disorder is selected from the group consisting of allergic, bacterial, chemical or viral conjunctivitis; blepharitis; dry eye syndrome; sub-conjunctival hematomas; corneal abrasion; uveitis; and combinations thereof.

### 5. BRIEF DESCRIPTION OF THE FIGURES

FIG. 1 illustrates a Layer by Layer coating procedure performed on an exemplary biomaterial.

FIG. 2A provides a photograph showing multiple corneal erosions in a patient with dry eyes. FIG. 2B illustrates a photograph showing confluent punctate epithelial erosions in a patient with dry eyes.

FIG. 3 shows a schematic diagram of lenses which are dipped into oppositely charged polymer solutions with and without complexation to an M2 polarizing agent.

FIG. 4A provides a photograph showing Senofilcon A lenses with/without alcian blue staining. FIG. 4B provides a photograph showing Lotrafilcon B lenses with/without alcian blue staining.

FIG. 5 provides a graph illustrating the cumulative release of IL-4 from lenses in varying conditions.

FIG. 6 provides a schematic diagram of an alternative Layer by Layer coating procedure performed on a contact lens.

FIG. 7A provides a graph illustrating the cumulative release of IL-4 from various types of lenses. FIG. 7B provides fluorescent images of intracellular arginase. FIG. 7C provides graphs showing the activity of arginase and the production of nitric oxide.

FIG. 8 provides scanning electron microscopy images of various types of lenses.

FIG. 9A provides a cross-sectional view of uncoated lenses. FIG. 9B provides a frontal view of uncoated lenses. FIG. 9C provides a cross-sectional view of coated lenses. FIG. 9D provides a frontal view of coated lenses. FIG. 9E provides a high-magnification cross-section view of coated lenses. FIG. 9F provides a high-magnification frontal view of coated lenses.

### 6. DETAILED DESCRIPTION OF THE INVENTION

The presently disclosed subject matter provides biomaterials coated with an active agent eluting coating (e.g., coated contact lens), wherein placement or implantation of the coated biomaterial results in modulation of the local immune reaction and reduced inflammation-related complications in eyes. In certain non-limiting embodiments, the biomaterials are coated with at least one polycation layer, at least one polyanion layer. In certain non-limiting embodiments, at least one layer of the coating contains an M2 polarizing active agent. The presently disclosed subject matter further provides methods for treating ocular disorders.

For clarity of description, and not by way of limitation, the detailed description of the invention is divided into the following subsections:

6.1 Definitions;

6.2 Coated biomaterial;

6.3 Methods of coating the biomaterial; and

6.4 Methods of treating ocular disorders.

### 6.1. Definitions

As used herein, the following terms have the meanings ascribed to them below, unless specified otherwise. Abbreviations used herein have their conventional meaning within the chemical and biological arts.

As used in the specification and the appended claims, the singular forms “a,” “an,” and “the” include plural referents unless the context clearly dictates otherwise. Thus, for example, a reference to “a compound” includes mixtures of compounds.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “biomaterial,” refers to a material which has properties that are adequate for mammalian body reconstruction, medical device construction, and/or drug control/release devices or products. This term includes absorbable devices and products, absorbable fabrics or meshes, absorbable adhesives and absorbable drug control/release devices) as well as non-absorbable devices and products, (e.g., implantable repair, contact lens, or support meshes). The term “absorbable” as used herein refers to materials that will be degraded and subsequently absorbed by the body. The term “non-absorbable” as used herein refers to materials that will not be degraded and subsequently absorbed by the body.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “effective amount”, as used herein, refers to that amount of active agent sufficient to treat, prevent, or manage a disease. Further, a therapeutically effective amount with respect to the second targeting probe of the disclosure can mean the amount of active agent alone, or in combination with other therapies, that provides a therapeutic benefit in the treatment or management of the disease, which can include a decrease in severity of disease symptoms, an increase in frequency and duration of disease symptom-free periods, or a prevention of impairment or disability due to the disease affliction. The term can encompass an amount that improves overall therapy, reduces or avoids unwanted effects, or enhances the therapeutic efficacy of or synergies with another therapeutic agent.

The terms “macrophage polarization” or “polarization of macrophages”, as used interchangeably herein, refer to controlling the macrophage microenvironment to elicit a particular macrophage phenotype. Polarized macrophages can be broadly classified into two main phenotypes: 1) M1, which is pro-inflammatory and 2) M2, which is anti-inflammatory/regulatory. Materials which elicit improved or regenerative remodeling outcomes are associated with a shift from an initially M1 to a more M2 profile during the early stages of the inflammatory response which follows implantation. Macrophages can be polarized to M2 by treating the microenvironment with M2 polarizing active agents such as IL-4, IL-13, IL-10, or combinations thereof and/or glucocorticoids.

The term “polyelectrolyte layers” refers to coating layers that are charged. For example, the polyelectrolyte layer can be either a polycation layer or a polyanion layer. A “polyelectrolyte bilayer” refers to a combination of a polycation and a polyanion polymer in a bilayer.

The term “polycation” refers to any polymer that has a net positive charge at the pH the layer is formed. Examples of polycations include, but are not limited to, a polysaccharide, a protein, a synthetic polyamine, or a synthetic polymer or polypeptide. In certain embodiments, polycation polysaccharides bearing one or more amino groups can be used herein. In certain embodiments, the polycation is the natural polysaccharide chitosan. As used herein, the term “chitosan” refers to a linear polysaccharide composed of randomly distributed 6-(1-4)-linked D-glucosamine (deacetylated unit) and N-acetyl-D-glucosamine (acetylated unit). Chitosan is produced by the deacetylation of chitin. The term “chitosan” relates to chitosan, chitosan derivatives and mixtures of chitosan and chitosan derivatives (e.g., glycol-chitosan, amine-grafted chitosan, fluorescent-tagged chitosan). Similarly, the protein can be synthetic or naturally-occurring. In certain embodiments, the biodegradable polyamine is a synthetic random copolypeptide, synthetic polyamine such as poly(β-aminoesters), polyester amines, poly(disulfide amines), mixed poly(ester and amide amines), and peptide crosslinked polyamines. The polycation polymers can be branched, linear, or a combination thereof.

The term “polyanion” refers to any polymer that has a net negative charge at the pH the layer is formed. Examples of polyanions include, but are not limited to, a polysaccharide, a protein, or a synthetic polymer or polypeptide. In certain embodiments, the polyanion is a polysaccharide. Examples of polyanion polysaccharides useful herein include, but are not limited to, a hyaluronate, alginate, chondroitin sulfate, dermatan, dermatan sulfate, heparan sulfate, or any combination thereof. The polyanion polymers can be branched, linear, or a combination thereof.

Ranges disclosed herein, for example “between about X and about Y” are, unless specified otherwise, inclusive of range limits about X and about Y as well as X and Y.

The terms “tune” or “tunable”, as used herein, means the ability to adjust the number and/or composition of layers of the coated biomaterial to alter the pharmacokinetic distribution of the active agent. For example, altering the number and/or composition of layers can alter active agent release characteristics such as, but not limited to, the dosage, release rates, duration, and distribution of the active agent. Tuning of the number and/or composition of layers of the coated biomaterial can be accomplished a number of ways, including but not limited to varying the number of alternating polycation and polyanion layers (e.g., increasing or decreasing the number of layers) prior to adding the active agent-containing layers; varying the polycation and/or polyanion used in the alternating polycation and polyanion layers (e.g. change the polycation and/or polyanion used or utilize different combinations of polycations and/or polyanions); altering the number of active agent-containing layers; altering the composition of the active agent-containing layer; adding additional polycation and/or polyanion layers on top and/or in between the active agent layers.

The term “active agent” refers to an agent that is capable of having a physiological effect when administered to a subject. In certain embodiments, the term “M2 polarizing active agent” refers to an agent that can polarize macrophages away from the M1 phenotype and/or towards the M2 phenotype, including for example, but not limited to, cytokines (e.g., IL-4, IL-13, IL-10, or combinations thereof), glucocorticoids (e.g., betamethasone, clocortolone, cortisone, dexamethasone, fludrocortisone, fluocortolone, fluprednylidene, hydrocortisone, medrysone, methylprednisolone, paramethasone, prednisolone, prednisone, prednylidene, triamcinolone, triamcinolone acetonide and their esters). In certain embodiments, the agent can be a protein (e.g., transcription factor), an antibiotic, a microRNA or combinations thereof.

A “subject” may be a human or a non-human animal, for example, but not by limitation, a non-human primate, a dog, a cat, a horse, a rodent, a cow, a goat, a rabbit, a mouse, etc.

The term “dosage” is intended to encompass a formulation expressed in terms of total amounts for a given timeframe, for example as μg/kg/hr, μg/kg/day, mg/kg/day, or mg/kg/hr. The dosage is the amount of an ingredient administered in accordance with a particular dosage regimen. A “dose” is an amount of an agent administered to a mammal in a unit volume or mass, e.g., an absolute unit dose expressed in mg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning in any case will be apparent from the context.

As used herein, “ocular disorder” “ophthalmic disease,” “ophthalmic disorder,” and the like, includes, but is not limited to, glaucoma, cataracts, leucoma, or retinal degeneration in a subject in need of such treatment comprising administering, to the subject, an effective amount of a compound as set forth above.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either endpoint of the range are specifically contemplated. For example, a nested sub-range of an exemplary range of 1 to 50 can include 1 to 10, 1 to 20, 1 to 30, and 1 to 40 in one direction, or 50 to 40, 50 to 30, 50 to 20, and 50 to 10 in the other direction.

The terms “treat,” “treating” or “treatment,” and other grammatical equivalents as used herein, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

### 6.2. Coated Biomaterials

The presently disclosed subject matter provides biomaterials coated with an active agent eluting coating. In certain non-limited embodiments, implantation of the coated biomaterial results in modulation of the local immune reaction and reduced inflammation-related complications in eyes as compared to non-coated biomaterials or coated biomaterials not containing an active agent. In certain embodiments, the biomaterials are coated with at least one polycation layer and at least one polyanion layer, wherein the polycation layers and the plurality of polyanion layers alternate to form a plurality of bilayers. In non-limiting embodiments, at least one layer of the coating can include an M2 polarizing active agent. The number and sequence of layers can be modified in order to provide the desired amount and release time of active agent from the coated biomaterial.

In certain non-limiting embodiments, the active agent is released from the coating and polarizes macrophages away from an M1 phenotype and/or to an M2 phenotype.

In certain non-limiting embodiments, implantation of the coated biomaterial results in reduced implant-related complications as compared to non-coated or coated biomaterial not containing an active agent. For example, but not limited to, the coated biomaterials (e.g., contact lens) can modulate the local immune reaction and reduce inflammation-related complications in eyes. In some embodiments, the coated biomaterial results in the diminished formation of fibrotic capsule surrounding the implant, reduced biomaterial associated inflammation and tissue degradation, and improved tissue integration.

In certain non-limiting embodiments, implantation, placement, or insertion of the coated biomaterial can result in reduced damage, dryness, and/or irritation of the eye. For example, the coated biomaterial (e.g., contact lens) can sustain release of immune modifying agents which can reprogram M1 macrophages to the anti-inflammatory M2 phenotype resulting in mitigation of symptoms and causes of inflammatory eye diseases.

In certain non-limiting embodiments, application of the coated biomaterial results in improved dry eye symptoms.

### 6.2.1. Biomaterials

The presently disclosed subject matter provides for a coated biomaterial, wherein the biomaterial can be any material which has properties that are adequate for mammalian body reconstruction, medical device construction, and/or drug control/release devices or products as defined above. In certain non-limiting embodiments, the biomaterial can be, but is not limited to, mesh, sutures, wound dressings, intraocular lenses, contact lenses, decellularized matrices, and biosensors.

The biomaterial can be made of any lens-type materials that will accept a coating. In non-limiting examples, the biomaterial can be made of polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-.epsilon.-caprolactone, polyanhydrides; polyphosphazines, poly(vinyl alcohols), poly(alkylene oxides) particularly poly(ethylene oxides), poly(allylamines)(PAM), poly(acrylates), modified styrene polymers such as poly(4-aminomethylstyrene), pluronic polyols, polyoxamers, poly(uronic acids), poly(vinylpyrrolidone) and/or copolymers of the above.

In certain non-limiting embodiments, the biomaterial can be made of a material that can hold a charge and/or a net charge. The biomaterial can hold either a positive or negative charge. For example, if the biomaterial holds a negative charge, the first coating layer next to the biomaterial should be a positively charged layer, and if the biomaterial holds a positive charge, the first coating layer next to the biomaterial should be a negatively charged layer.

In certain non-limiting embodiments, the biomaterial can be, but is not limited to contact lens. The contact lens can be of any type, shape, size, or material. For example, but not limited to, the contact lens can be a rigid, soft, or hybrid contact lens. The rigid contact lens can be a gas permeable contact lens such as a porous polymethyl methacrylate (PMMA) lens. The soft contact lens can be made of soft, flexible plastics that allow oxygen to pass through to the cornea. For example, the soft contact lens can be a hydrogel contact lens or a silicone hydrogel contact lens. In certain embodiments, the silicone hydrogel contact lens can include balafilcon A, lotrafilcon A, lotrafilcon B, etafilcon A, Narafilcon A, galyfilcon A, senofilcon A, ocufilcon D, hioxifilicon A, enfilcon A, comfilcon A, nesofilcon A, filicon II 3, deleficon A, methafilcon A, methafilcon B, vifilcon A, phemfilcon A, nelfilcon A, stenfilcon A, polymacon, hefilcon B, tetrafilcon A, omafilcon A, balafilcon A, polymacon B, hilafilcon B, alphafilcon A, or combination thereof. Other examples of hydrogel contact lens can include tefilcon, lidofilcon B, etafilcon, bufilcon A, tetrafilcon A, surfilcon, bufilcon A, perfilcon, crofilcon, lidofilcon A, deltafilcon A, dimefilcon, ofilcon A, droxifilcon A, ocufiicon B, hefilcon A & B, xylofilcon A, phemfilcon A, phemfilcon A, phemfilcon A, scafilcon A, ocufiicon, tetrafilcon B, isofilcon, methafilcon, mafilcon, vifiicon A, polymacon or a combination of thereof. The hybrid contact lens can have a rigid gas permeable central zone, surrounded by the hydrogel or silicone hydrogel material.

The total number of layers of the biomaterial coating should not alter the architecture or topography of the biomaterial. For example, the biomaterial coating should not alter the biomaterial shape, size, performance, porosity, or combinations thereof.

In certain embodiments, the coating on the biomaterial can be from about 0.5 nm to about 500 μm thick. In certain embodiments, the coating on the biomaterial can be from about 1 nm to about 400 μm, about 10 nm to about 300 μm, about 20 nm to about 200 μm, about 30 nm to about 100 μm, about 40 nm to about 50 μm, about 50 nm to about 10 μm, about 60 nm to about 1 μm, about 70 nm to about 900 nm, about 80 nm to about 800 nm, about 90 nm to about 700 nm, about 100 nm to about 600 nm, about 200 nm to about 500 nm, or about 300 nm to about 400 nm thick. In certain embodiments, the coating can be from about 0.3 nm to about 0.8 nm, about 0.4 nm to about 0.7 nm, or about 0.5 nm to about 0.6 nm in thickness. In certain embodiments, the coating can be from about 1 nm to about 1000 nm, about 10 nm to about 900 nm, about 20 nm to about 800 nm, about 30 nm to about 700 nm, about 40 nm to about 600 nm, about 50 nm to about 500 nm, about 60 nm to about 400 nm about 70 nm to about 300 nm, about 80 nm to about 200 nm or about 90 nm to about 100 nm in thickness. In certain embodiments, the coating can be from about 1 μm to about 500 μm, about 10 μm to about 400 μm, about 20 μm to about 300 μm, about 30 μm to about 200 μm, about 40 μm to about 100 μm, about 50 μm, to about 90 μm, or about 60 μm to about 80 μm in thickness. In certain embodiments, the coating is no more than about 0.2 nm, about 0.3 nm, about 0.4 nm, about 0.5 nm, about 0.6 nm, about 0.7 nm, about 0.8 nm, about 0.9 nm, or about 1 nm in thickness. In certain embodiments, the coating is no more than about 2 nm, about 3 nm, about 4 nm, about 5 nm, about 6 nm, about 7 nm, about 8 nm, about 9 nm, about 10 nm, about 20 nm, about 30 nm, about 40 nm, about 50 nm, about 60 nm, about 70 nm, about 80 nm, about 90 nm, or about 100 nm in thickness.

### 6.2.2. Coating Layers

The presently disclosed subject matter provides various layers which can be coated on the disclosed biomaterial. In non-limiting embodiments, the coating layers can include at least one polyelectrolyte layer, at least one active agent containing layer, or a combination thereof.

Polyelectrolyte Layers

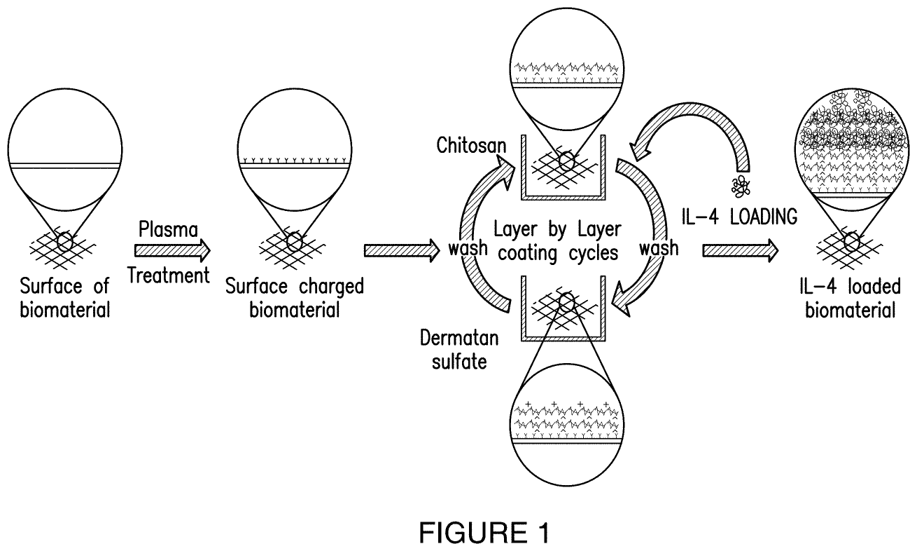

The presently disclosed subject matter provides for a coated biomaterial, wherein the biomaterial can be coated with polyelectrolyte layers. In certain non-limiting embodiments, the biomaterial can be coated with alternating polycation and polyanion layers (i.e., polyelectrolyte bilayers) (see FIG. 1). The layer closest to the biomaterial can be either a polycation layer or a polyanion layer. The layer closest to the active agent-containing layer can be either a polycation layer or a polyanion layer. In certain non-limiting embodiments, the polycation and/or polyanion layers can be distributed among and/or on top of the active agent-containing layers.

In certain non-limiting embodiments, the polyelectrolytes can be antimicrobial. The polycation can be a polysaccharide, a protein, a synthetic polyamine, or a synthetic polymer or polypeptide as discussed above. The polyanion can be a polysaccharide, a protein, or a synthetic polymer or polypeptide as discussed above. In certain non-limiting embodiments, as exemplified below, the polycation is chitosan. Chitosan has known biocompatibility, antimicrobial activity, and is degraded by activated macrophages. In certain non-limiting embodiments, as exemplified below, the polyanion is dermatan sulfate. Dermatan sulfate (also known as chondroitin sulfate B) plays a role in extracellular matrix (ECM) regulation and is able to enhance IL-4 bioactivity in-vivo. In certain non-limiting embodiments, the alternating polycation and polyanion layers can be chitosan-dermatan sulfate alternating layers. In certain embodiments, the alternating chitosan-dermatan sulfate alternating layers can provide enhanced release and bioactivity of the active agent (e.g., IL-4) in the context of macrophage mediated host-implant interactions.

In certain embodiments, the total number of alternating polycation and polyanion layers can be adjusted in order to tune the release of the active agent from the coated biomaterial. In certain embodiments, the bilayer core coating serves to make the surface charge more solid, consistent, and/or strong for the deposition of the active agent containing layers.

In certain non-limiting embodiments, the total number of alternating polycation and polyanion layers (i.e., polyelectrolyte bilayers) can be from about 10 to about 1000 bilayers. In certain embodiments, the total number of polyelectrolyte bilayers can be from about 20 to about 900, about 30 to about 800, about 40 to about 700, about 50 to about 600, about 60 to about 500, about 70 to about 400, about 80 to about 300, or about 90 to about 200. In certain embodiments, the total number of polyelectrolyte bilayers can be from about 20 to about 90, about 30 to about 80, about 40 to about 70, or about 50 to about 60. In certain embodiments, the total number of polyelectrolyte bilayers can be from about 6 to about 18 bilayers, about 8 to about 16 bilayers, or about 10 to about 14 bilayers. In certain embodiments, the total number of polyelectrolyte bilayers can be from about 8 to about 14 bilayers or about 10 to about 12 bilayers. In certain embodiments, the total number of polyelectrolyte bilayers can be at least 4 bilayers, at least 6 bilayers, at least 8 bilayers, at least 10 bilayers, at least 12 bilayers, at least 14 bilayers, at least 16 bilayers, at least 18 bilayers, or at least 20 bilayers. In certain embodiments, the total number of polyelectrolyte bilayers can be about 10 bilayers.

The polycation layer can be made of one type of polycation or a combination of different polycations. In certain embodiments, each polycation layer contains only one type of polycation. In certain non-limiting embodiments, the coated biomaterial contains more than one type of polycation, wherein the different polycations are in the same and/or different layers.

The polyanion layer can be made of one type of polyanion or a combination of different polyanions. In certain non-limiting embodiments, each polyanion layer contains only one type of polyanion. In certain non-limiting embodiments, the coated biomaterial contains more than one type of polyanion, wherein the different polyanions are in the same and/or different layers.

In certain non-limiting embodiments, the polyelectrolyte layer contains additional excipients known to those of skill in the art.

In certain non-limiting embodiments, the polyelectrolyte layer can be coated on a surface of biomaterial (e.g., contact lens) through a Layer bu Layer (LbL) procedure. For example, the biomaterial can undergo alternating immersion into polycation and polyanion solutions (e.g., about 1 mg/mL, about 2 mg/mL, about 5 mg/mL, about 10 mg/mL, about 50 mg/mL, or about 100 mg/mL) for about 1 minute, about 5 minutes, about 10 minutes, about 15 minutes, about 30 minutes, about 60 minutes, about 120 minutes, or about 180 minutes at room temperature.

Active Agent Containing Layer

The presently disclosed subject matter provides for a coated biomaterial, wherein the biomaterial can be further coated with an active agent containing layer. In certain non-limiting embodiments, the biomaterial coated with the alternating polycation and polyanion layers can be further coated with at least one active agent containing layer (see FIG. 1). The polyelectrolyte layer closest to the active agent containing layer can be either a polycation layer or a polyanion layer. In certain non-limiting embodiments, the polyelectrolyte layers can be distributed among and/or on top of the active agent containing layers.

In certain non-limiting embodiments, the active agent containing layer can include either a polycation or a polyanion. In certain embodiments, if the active agent containing layer holds a negative charge, the polyelectrolyte layer next to the active agent containing layer should be a polycation layer, and if the active agent containing layer holds a positive charge, the polyelectrolyte layer next to the active agent containing layer should be a polyanion layer.

Materials which elicit improved or regenerative remodeling outcomes are associated with a shift from an initially M1 to a more M2 profile during the early stages of the inflammatory response which follows insertion or implantation of the coated biomaterials. In addition, dry eye disease is known to include a self-perpetuating cycle of inflammation, with the M1 macrophages as the major mediator and driver. In certain embodiments, the active agent is one that can mitigate symptoms and causes of inflammatory ocular disorders. For example, the active agent can polarize macrophages away from the M1 phenotype and/or towards the M2 phenotype. In certain embodiments, polarization of the macrophages to the M2 phenotype will alleviate a symptom of the ocular disorders, wherein the symptom comprises redness, itching, burning, foreign body sensation, watery eyes, dry eyes, swelling, pain, clouding of vision, secretion of pus, sticking eyelids, altered sensitivity to light, or a combination of thereof. In a non-limiting embodiment, the ocular disorders can be allergic, bacterial, chemical or viral conjunctivitis, blepharitis, dry eye syndrome, sub-conjunctival hematomas, corneal abrasion, uveitis, or combinations thereof.

The macrophage phenotype can be tested by immunocytochemistry. For example, immuno-labeling can be performed to assess the phenotypic profiles of the cells of the surface of the eye after coated contact lens wear. The presence of arginase-1 (an M2 marker) and/or inducible nitric oxide synthase (iNOS, an M1 marker) can be assessed. Image analysis can be performed using a custom-designed algorithm (Wolfram Mathematica, Version 10.0) in order to quantify labeling (normalized and expressed as cumulative arginase-1/DAPI pixel ratio) of cells in the eye.

The total number of active agent containing layers can be adjusted in order to tune the pharmacokinetic profile of the active agent from the coated biomaterial. In certain embodiments, the total number of active agent containing layers can be from about 10 to about 1000 bilayers. Depending on what polyelectrolyte is contained in the active agent layer, each active agent layer is separated by a polyelectrolyte layer of the opposite charge with or without an active agent. In certain embodiments, the total number of active agent containing layers can be from about 20 to about 900 layers, about 30 to about 800 layers, about 40 to about 700 layers, about 50 to about 600 layers, about 60 to about 500 layers, about 70 to about 400 layers, about 80 to about 300 layers, or about 90 to about 200 layers. In certain embodiments, the total number of active agent containing layers can be from about 20 to about 90 layers, about 30 to about 80 layers, about 40 to about 70 layers, or about 50 to about 60 layers. In certain embodiments, the total number of active agent containing layers can be from about 10 to about 75 layers, about 15 to about 60 layers, about 20 to about 55 layers, about 25 to about 50 layers, about 30 to about 45 layers or about 35 to about 40 layers. In certain embodiments, the total number of active agent containing layers can be from about 20 to about 30 layers, about 20 to about 40 layers, about 20 to about 50 layers, about 20 to about 60 layers, about 30 to about 40 layers, about 30 to about 50 layers, about 30 to about 60 layers, about 40 to about 50 layers, about 40 to about 60 layers, or about 50 to about 60 layers. In certain embodiments, the total number of active agent containing layers can be from about 22 to about 38 layers, about 24 to about 36 layers, about 26 to about 34 layers, about 28 to about 32 layers, about 32 to about 48 layers, about 34 to about 46 layers, about 36 to about 44 layers, about 38 to about 42 layers, about 42 to about 58 layers, about 44 to about 56 layers, about 46 to about 54 layers, or about 48 to about 52 layers. In certain embodiments, the total number of active agent containing layers can be at least 20 layers, at least 21 layers, at least 22 layers, at least 23 layers, at least 24 layers, at least 25 layers, at least 26 layers, at least 27 layers, at least 28 layers, at least 29 layers, at least 30 layers, at least 31 layers, at least 32 layers, at least 33 layers, at least 34 layers, at least 35 layers, at least 36 layers, at least 37 layers, at least 38 layers, at least 39 layers, at least 40 layers, at least 41 layers, at least 42 layers, at least 43 layers, at least 44 layers, at least 45 layers, at least 46 layers, at least 47 layers, at least 48 layers, at least 49 layers, at least 50 layers, at least 51 layers, at least 52 layers, at least 53 layers, at least 54 layers, at least 55 layers, at least 56 layers, at least 57 layers, at least 58 layers, at least 59 layers, or at least about 60. In certain embodiments, the total number of active agent containing layers can be about 20 layers, about 21 layers, about 22 layers, about 23 layers, about 24 layers, about 25 layers, about 26 layers, about 27 layers, about 28 layers, about 29 layers, about 30 layers, about 31 layers, about 32 layers, about 33 layers, about 34 layers, about 35 layers, about 36 layers, about 37 layers, about 38 layers, about 39 layers, about 40 layers, about 41 layers, about 42 layers, about 43 layers, about 44 layers, about 45 layers, about 46 layers, about 47 layers, about 48 layers, about 49 layers, about 50 layers, about 51 layers, about 52 layers, about 53 layers, about 54 layers, about 55 layers, about 56 layers, about 57 layers, about 58 layers, about 59 layers, about 60, about 61, about 62, about 63, about 64, about 65, about 66, about 67, about 68, about 69, about 70, about 71, about 72, about 73, about 74, about 75, about 76, about 7, about 78, about 79, or about 80.

The total number of polyelectrolyte and/or active agent containing layers can be adjusted in order to tune the release of the active agent from the coated biomaterial. For example, the composition of the layers can be altered such that the dosage, release rates, duration, and distribution of the active agent can be controlled.

In certain non-limiting embodiments, the polyelectrolyte and/or active agent containing layers can be adjusted so that the active agent is released to provide an effective concentration of the active agent at a distance of about 10 μm to about 100 μm from the coated biomaterial. In certain embodiments, the layers can be adjusted so that the active agent is released at a distance of about 15 μm to about 95 μm, about 20 μm to about 90 μm, about 25 μm to about 85 μm, about 30 μm to about 80 μm, about 35 μm to about 75 μm, about 40 μm to about 70 μm, about 45 μm to about 65 μm, or about 50 μm to about 60 μm from the coated biomaterial. In certain embodiments, the layers can be adjusted so that the active agent is released at a distance of about 5 μm to about 50 μm, about 10 μm to about 45 μm, about 15 μm to about 40 μm, about 20 μm to about 35 or about 25 μm to about 30 μm. In certain embodiments, the layers can be adjusted so that the active agent is released at a distance of about 50 μm from the coated biomaterial.

In certain embodiments, the layers can be adjusted so that the active agent is released to provide an effective concentration of the active agent at a distance of up to about 100 μm, up about 95 μm, up to about 90 μm, up to about 85 μm, up to about 80 μm, up to about 75 μm, up to about 70 μm, up to about 65 μm, up to about 60 μm, up to about 55 μm, or up to about 50 μm from the coated biomaterial. As used herein, the phrase “effective concentration” means a concentration of the active agent that is able to polarize macrophages towards the M2 phenotype and away from the M1 phenotype. In certain embodiments, the phrase “effective concentration” also means a concentration of the active agent that is able to alleviate a symptom of the ocular disorders, wherein the symptom comprises redness, itching, burning, foreign body sensation, watery eyes, dry eyes, swelling, pain, clouding of vision, secretion of pus, sticking eyelids, altered sensitivity to light, or a combination of thereof.

In certain non-limiting embodiments, the polyelectrolyte and/or active agent containing layers can be adjusted so that the active agent is released for about 2 days to about 14 days, about 2 days to about 7 days, about 2 days to about 10 days, about 7 days to about 14 days, or about 7 days to about 10 days. In certain embodiments, the polyelectrolyte and/or active agent containing layers can be adjusted so that the active agent is released for at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days. In certain embodiments, the polyelectrolyte and/or active agent containing layers can be adjusted so that the active agent is released for no more than about 2 days, no more than about 3 days, no more than about 4 days, no more than about 5 days, no more than about 6 days, no more than about 7 days, no more than about 8 days, no more than about 9 days, no more than about 10 days, no more than about 11 days, no more than about 12 days, no more than about 13 days, no more than about 14 days, no more than about 15 days, no more than about 16 days, at least no more than about 17 days, at least no more than about 18 days, at least no more than about 19 days, or at least no more than about 20 days.

In certain non-limiting embodiments, the active containing layer can be coated on a biomaterial or another layer through a LbL coating procedure. For example, but not by way of limitation, the active agent IL-4 (e.g., about 0.5 μg/mL, about 1 μg/mL, about 1.5 μg/mL, about 3 μg/mL, about 5 μg/mL, about 10 μg/mL, about 15 μg/mL, about 30 μg/mL, about 50 μg/mL, about 100 μg/mL, about 500 μg/mL, about 1000 μg/mL, about 2500 μg/mL, or about 5000 μg/mL) can be complexed with dermatan sulfate (e.g., about 1 mg/mL, about 2 mg/mL, about 5 mg/mL, about 10 mg/mL, about 15 mg/mL, about 20 mg/mL, about 50 mg/mL, about 100 mg/mL, about 250 mg/mL, about 500 mg/mL, about 1000 mg/mL, about 2500 mg/mL, or about 5000 mg/mL) by incubating the mixture overnight at 4° C. Then, the biomaterial (e.g., contact lens) can be further coated with at least one layer containing the active agent using the LbL method. After coating, the active agent coated loaded lens can be lyophilized and stored at −20° C. or can be placed in a humidified chamber and stored at 4° C.

In certain non-limiting embodiments, the biomaterial coating provides for a delay in the release of the active agent from the coating. The delay in the release can occur by coating non-active agent containing layers on top of the active agent containing layers. The number of layers can be adjusted to tune the release of the active agent from the biomaterial coating.

The M2 polarizing active agent can be a cytokine such as IL-4, IL-13, and IL-10 or glucocorticoids such as betamethasone, clocortolone, cortisone, dexamethasone, fludrocortisone, fluocortolone, fluprednylidene, hydrocortisone, medrysone, methylprednisolone, paramethasone, prednisolone, prednisone, prednylidene, triamcinolone, triamcinolone acetonide and their esters. In non-limiting embodiments, the active agent can be a protein (e.g., transcription factor), an antibiotic, a microRNA, or combinations thereof. In certain non-limiting embodiments, as exemplified below, the active agent can be IL-4. In certain embodiments, the cytokine is complexed to the polyelectrolyte, and the concentration of the cytokine is dependent on the ratio of cytokine complexed with the polyelectrolyte and the number of active agent containing layers. For example, IL-4 can be complexed with dermatan sulfate at a particular ratio (e.g., 1.5 μg/mL IL-4 to 2 mg/mL dermatan sulfate). In certain embodiments, the active agents can be complexed into certain polymers based on electrostatic interactions. For example, IL-4 can be complexed into dermatan sulfate, as it has a relatively high isoelectric point and thus can be positively charged at physiologic pH, rendering it attracted to negatively charged groups in dermatan sulfate. In addition, dermatan sulfate is a naturally occurring biopolymer present in the extracellular matrix and can facilitate, enhance, and support certain cellular signaling functions of IL-4. In certain embodiments, the ratio of active agent to dermatan sulfate can be from about 1:2000 to about 1:10. In certain embodiments, the ratio of active agent to dermatan can be from about 1:1900 to about 1:20, about 1:1800 to about 1:30, about 1:1700 to about 1:40, about 1:1600 to about 1:50, about 1:1500 to about 1:60, about 1:1400 to about 1:70, about 1:1300 to about 1:80, about 1:1200 to about 1:90, about 1:1100 to about 1:100, about 1:1000 to about 1:200, about 1:900 to about 1:300, about 1:800 to about 1:400, or about 1:700 to about 1:500.

The active agent containing layer can include one type of active agent or a combination of different active agents. In certain embodiments, each active agent containing layer contains only one type of active agent. In certain embodiments, the active agent containing layer contains more than one type of active agent, wherein the different active agents are in the same and/or different layers.

In certain non-limiting embodiments, the active agent containing layer contains additional excipients. For example, the active agent containing layer can contain a polycation and/or polyanion. In certain non-limiting embodiments, as exemplified below, the active agent can be IL-4 in combination with dermatan sulfate.

In certain non-limiting embodiments, the degradation of the polyelectrolyte layer and/or release rate of active agents can be adjusted by macrophage-related enzymes. The macrophage-related enzymes mimic those produced by macrophages. For example, but not limited to, the coated contact lens can include the enzymes or be inserted with the enzymes. These enzymes can include, but are not limited to, chitosanase, chondroitinase, matrix metalloproteinases, collagenase, or a combination thereof. In non-limiting embodiments, certain enzymes (e.g., chitosanase, chondroitinase, or lysozyme) can expedite the degradation of the coating. In some embodiments, the disclosed enzymes can mimic bioactivities of enzymes found naturally in a body.

### 6.2.3. Coated Contact Lens

In non-limiting embodiments, the presently disclosed subject matter can provide a contact lens which can be coated with the disclosed coating layers. For example, but not by way of limitation, the contact lens can be coated with at least one polycation layer, polyanion layer, active agent containing layer, or combinations thereof. In certain non-limiting embodiments, the polycation can be, but is not limited to, one or more polysaccharide (e.g., chitosan). In certain non-limiting embodiments, the polycation (which can be comprised of one or more species of cation) can be, but is not limited to, one or more protein (e.g., collagen), a synthetic polyamine, or positively charged polymers or copolymers.

In certain non-limiting embodiments, the polyanion (which can be comprised of one or more species of anion) in at last one polyanion layer is selected from the group consisting of glycosaminoglycan (e.g., dermatan, dermatan sulfate, hyaluronate, an alginate, chondroitin sulfate, heparan sulfate, or any combination thereof or negatively charged polymers or copolymers (e.g., polyacrylates, polyesters, polyurethanes). The polyanion (which can be comprised of one or more species of anion) can be, but is not limited to, glycosaminoglycan (e.g., dermatan or dermatan sulfate).

In certain non-limiting embodiments, the active agent can be a cytokine (e.g., IL-4, IL-13, IL-10, TGF-β, HGF or combinations thereof), one or more glucocorticoid or combination of glucocorticoids. In another embodiment, the active agent can be a protein (e.g., transcription factor), an antibiotic, a microRNA or combinations thereof. In certain embodiments, wherein the plurality of polycation layers and the plurality of polyanion layers alternate to form a plurality of bilayers. In non-limiting embodiments, at least one layer of the coating comprises dermatan sulfate and the M2 polarizing active agent. The M2 polarizing active agent and the dermatan sulfate can be present in a ratio between about 1:10 to about 1:2000. In certain embodiments, the thickness of the coating is between about 0.5 nm and about 500 μm. In certain non-limiting embodiments, the disclosed LbL coating technique can provide a relatively thin coating (e.g., from about 0.5 nm to about 1000 nm) on a biomaterial. The thin coating can allow the biomaterial to preserve its unique properties (e.g., optical properties) in the presence of the coating. For example, the coating layer can be placed on a contact lens without altering its optical property (e.g., vision correction). The coating layer can be also degraded without altering the contact lens' optical property. Accordingly, the disclosed contact lens can simultaneously correct the vision of a subject during the release of active agents from the coating.

In certain non-limiting embodiments, the lens body can be a silicone hydrogel contact lense. The contact lens can be selected from the group consisting of balafilcon A, lotrafilcon A, lotrafilcon B, etafilcon A, narafilcon A, galyfilcon A, senofilcon A, ocufilcon D, hioxifilicon A, enfilcon A, comfilcon A, nesofilcon A, filicon II 3, deleficon A, methafilcon A, methafilcon B, vifilcon A, phemfilcon A, nelfilcon A, stenfilcon A, polymacon, hefilcon B, tetrafilcon A, omafilcon A, polymacon B, hilafilcon B, alphafilcon A, and combination thereof.

In certain non-limiting embodiments, the coated contact lens can include an enzyme. The enzyme can include a macrophage-related enzyme. The macrophage-related enzyme can degrade the coating and control the release rate of an active agent from the coating. In certain embodiments, the active agent is released from the coating to polarize macrophages to an M2 phenotype and to provide an effective concentration of the active agent in a predetermined distance from the surface of the coating.

In certain embodiments, as opposed to a quick burst release of drug (as seen with eye drops), the coated contact lens can sustain the release of active agent for at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days.

### 6.3. Methods of Coating the Biomaterial

The presently disclosed subject matter also relates to methods for coating the biomaterial. In certain non-limiting embodiments, the biomaterial can be negatively or positively charged or treated such that the surface becomes negatively or positively charged. The coating process can occur by alternate cyclic deposition of multiple polyelectrolyte layers mediated by opposite electrostatic charges on the surface of a charged substrate.

In certain non-limiting embodiments, the negatively charged biomaterial can be coated with a polycation layer. In certain embodiments, the positively charged biomaterial can be coated with a polyanion layer. In certain non-limiting embodiments, the biomaterial can be coated with alternating polycation and polyanion layers. In certain non-limiting embodiments, once the biomaterial is coated with alternating polycation and polyanion layers, the coated biomaterial can be coated with at least one layer containing at least one active agent. In certain non-limiting embodiments, polycation and/or polyanion layers can be among and/or on top of the active agent containing layer(s). In certain embodiments, the coated biomaterial can be sterilized.

In certain non-limiting embodiments, the surface of the biomaterial is cleaned prior to the addition of a charge or any of the coating layers. For example, the surface of the biomaterial can be cleaned with a solution of water, acetone, isopropanol, ethanol, methanol, benzene, hydrogen peroxide, dioxane, tetrahydrofuran or combinations thereof.

In certain non-limiting embodiments, as exemplified below, the biomaterial can become charged prior to the application of the coating. The biomaterial can be irradiated to form a consistent and durable charge on the surface of the biomaterial. For example, the biomaterial can be irradiated with radiofrequency glow discharge (RFGD) or plasma-enhanced chemical vapor deposition (PECVD) to form either a negative or positive charge on the surface of the biomaterial.

In an alternative embodiment, the biomaterial is already charged and can be coated without receiving the plasma treatments.

In certain non-limiting embodiments, the polycation can be dissolved in a suitable solvent or buffer known to those of skill in the art for the particular polycation. In certain embodiments, the polyanion can be dissolved in a suitable solvent or buffer known to those of skill in the art for the particular polyanion. Suitable solvent include, but are not limited to, water and acetate, phosphate, saline buffer, acetic acid, hydrochloric acid, methanol, isopropanol, ethanol, n-propanol, n-butanol, isobutanol, t-butanol, dimethyl sulfoxide, N, N-dimethylformamide, N, N-dimethylacetamide, methyl acetate, ethyl acetate, isopropyl acetate, acetone, methyl ethyl ketone, methyl isobutyl ketone, or combinations thereof.

The biomaterial can then be soaked in a solution of the polyelectrolyte and then washed in water or buffer and allowed to dry. The coated biomaterial can then be soaked in a solution of polyelectrolyte of an opposite charge, washed in water or buffer and allowed to dry. In certain embodiments, the drying process can utilize pressurized clean air. This process can continue until the appropriate number of layers is added to the biomaterial.

In certain non-limiting embodiments, as exemplified below, the negatively charged biomaterial can be dipped in a chitosan solution for 10 minutes at room temperature, then washed three times in milli-Q water and air dried, and once dried, the biomaterial can be dipped in a dermatan sulfate solution for 10 minutes at room temperature, then washed three times in milli-Q water and air-dried.

In certain non-limiting embodiments, once the biomaterial is coated with the appropriate amount of polyelectrolyte layers, it can be coated with an active agent containing layer. In certain embodiments, the active agent containing layer is coated on top of the biomaterial without the polyelectrolyte layers. The coated or uncoated biomaterial can be soaked in the active agent containing solution and then washed in water or buffer and allowed to dry. Depending on the polyelectrolyte present in an active agent containing layer, the biomaterial is next soaked in a polyelectrolyte solution (with or without an active agent) of the opposite charge and then washed in water or buffer and allowed to dry. This process can continue until the appropriate amount of active agent containing layers is added to the biomaterial.

In certain non-limiting embodiments, as exemplified below, the biomaterial can be dipped in a solution of IL-4-dermatan sulfate mixture for 10 minutes at room temperature, then washed three times in milli-Q water and air dried, and once dried, the biomaterial can be dipped in a chitosan solution for 10 minutes at room temperature, then washed three times in milli-Q water and air-dried.

In certain non-limiting embodiments, the coated biomaterial can be sterilized using ethylene oxide gas, gamma irradiation, and E-beam sterilization. In certain embodiments, the contact lens can be sterilized without clattering an architecture or a topography of the coating.

In certain non-limiting embodiments, the method of coating the biomaterial can be as follows. For this illustration, the biomaterial is a contact lens, but any biomaterial can be used. The method of coating the contact lens can entail washing the biomaterial with a cleaning solution, such as, but not limited to water, acetone, isopropanol, ethanol, methanol, benzene, hydrogen peroxide, dioxane, tetrahydrofuran or combinations thereof (e.g., a 1:1 acetone:isopropanol mixture) followed by air drying. The washed lens can then be further cleaned to, for example, remove any organic contamination by any method known by those of skill in the art. For example, the washed lens can be irradiated with gas plasma, such as but not limited to argon plasma (e.g., at 600 W with a gas flow of 35 mL/min with a steady pressure of 250 mTorr). Once clean, the lens can be treated to obtain a negatively charged surface if the surface is not already inherently charged. For example, the cleaned lens can be exposed to an adapted radio frequency glow discharge (RFGD) via a microwave plasma procedure (e.g., maleic anhydride can be used as a monomer for RFGD treatments followed by hydrolysis). In certain non-limiting embodiments, the lens can be washed with water and boiled in freshwater prior to the coating process. In order to deposit a conformal coating onto the surface of the negatively charged lens, a Layer by Layer (LbL) procedure can be performed. The charged lens can undergo alternating immersion into polycation and polyanion solutions (e.g., 2 mg/mL, 10 minutes each at room temperature) with intermediate washings in water. For example, the polycation can be chitosan (dissolved in 0.5% acetic acid for example) and the polyanion can be dermatan sulfate (dissolved in water). First, the lens can be dipped in the polycation solution for 10 minutes at room temperature, then washed (e.g., 3 times—10, 20 and 30 seconds—in milli-Q water) and air-dried (e.g., using pressurized clean air). Next, the lens can be dipped in a polyanion solution for 10 minutes at room temperature. The lens can then be washed again in milli-Q water and air-dried. This coating cycle can be repeated until a core coating of bilayers is achieved (e.g., 10). After coating, the lens can be either lyophilized or not lyophilized and stored at 4° C. in a humidified or non-humidified environment. Next, the core coated lens can be coated with the active agent. For example, but not by way of limitation, the active agent IL-4 (e.g., 1.5 μg/mL) can be complexed with dermatan sulfate (e.g., 2 mg/mL) by incubating the mixture overnight at 4° C. Then, the coated lens can be further coated with 20, 40 and 60 bilayers containing the active agent using the same LbL method used for the core coating. After coating, the active agent coated loaded lens can be lyophilized and stored at −20° C.

In certain non-limiting embodiments, the coating can be applied in a uniform way to the surface of the biomaterial. To visualize coating adherence, glycosaminoglycan coating components can be stained with an alcian blue dye to confirm that the coating is uniformly applied to the lens.

In certain embodiments, the disclosed LbL coating technique can provide a relatively thin coating (e.g., from about 1 nm to about 1000 nm) on a biomaterial. The thin coating can allow the biomaterial to preserve its unique properties (e.g., optical properties) in the presence of the coating. For example, the coating layer can be placed on a contact lens without altering its optical property (e.g., vision correction). The coating layer can be also degraded without altering the contact lens' optical property. Accordingly, the disclosed contact lens can simultaneously correct the vision of a subject during the release of active agents from the coating.

### 6.4. Methods of Treating Ocular Disorders

The presently disclosed subject matter provides a method for treating ocular disorders. In certain non-limiting embodiments, the method comprises placing a coated contact lens on a surface of an eyeball. In certain embodiments, the coating on the surface of the contact lens can include a plurality of polycation layers and a plurality of polyanion layers, wherein the plurality of polycation layers and the plurality of polyanion layers alternate to form a plurality of bilayers. In non-limiting embodiments, at least one layer of the coating includes an M2 polarizing active agent.

In certain non-limiting embodiments, implantation or insertion of the coated biomaterial can result in reduced damage, dryness, and/or irritation of the eye. For example, the coated biomaterial (e.g., contact lens) can sustain the release of immune modifying agents which can reprogram M1 macrophages to the anti-inflammatory M2 phenotype resulting in mitigation of symptoms and causes of inflammatory eye diseases. The symptoms of inflammatory eye diseases include redness, itching, burning, foreign body sensation, watery eyes, dry eyes, swelling, pain, clouding of vision, secretion of pus, sticking eyelids, and/or altered sensitivity to light. In non-limiting embodiments, the effective concentration can be a concentration of the active agent that alleviates a symptom of the ocular disorders.

In certain embodiments, the inflammatory eye disease can include dry eye syndrome, uveitis, scleritis, keratoconjunctivitis sicca (KCS), Sjogren syndrome (SS), Sjogren syndrome associated keratoconjunctivitis sicca, non-Sjogren syndrome associated keratoconjunctivitis sicca, keratitis sicca, sicca syndrome, xerophthalmia, tear film disorder, aqueous tear deficiency (ATD), meibomian gland dysfunction (MGD), ocular tumors, neovascular proliferative diseases, neovascular maculopathies, rheumatoid corneal melting disorders, autoimmune disorders, orbital inflammatory disease, diabetic retinopathies, proliferative retinopathies, retinopathy of prematurity, retinal vascular diseases, vascular anomalies, age-related macular degeneration and other acquired disorders, endophthalmitis, infectious diseases, inflammatory diseases, AIDS-related disorders, ocular ischemia syndrome, pregnancy-related disorders, peripheral retinal degenerations, retinal degenerations, toxic retinopathies, cataracts, retinal tumors, corneal neovascularization, choroidal tumors, choroidal disorders, choroidal neovascularization, neovascular glaucoma, vitreous disorders, retinal detachment and proliferative vitreoretinopathy, cyclitis, non-penetrating trauma, penetrating trauma, post-cataract complications, Hippel-Lindau Disease, inflammatory optic neuropathies, macular edema, pterygium, iris neovascularization, and/or surgical-induced disorders.

In certain non-limiting embodiments, the coated contact lens can be worn continuously for about 5 hours, about 10 hours, about 20 hours, about 24 hours, about 2 days, about 3 days, about 4 days, about 5 days, about 6 days, about 7 days, about 8 days, about 9 days, about 10 days, about 11 days, about 12 days, about 13 days, about 10 days, about 14 days, about 20 days, or about 30 days. In certain embodiments, the contact lens can be worn continuously for about 30 days.

In certain non-limiting embodiments, the method further includes delivering a supplement solution to the eyeball. In non-limiting embodiments, the supplement solution can include artificial tears and/or a macrophage-related enzyme. The supplement solution can be delivered simultaneously with the contact lens. In certain embodiments, the supplement solution and the contact lens can be delivered sequentially. In certain embodiments, the degradation of the coating and/or release rate of the active agents from the coating can be controlled by delivering various concentrations of the macrophage-related enzymes in the supplement solution. These enzymes can include, but are not limited to, chitosanase, chondroitinase, matrix metalloproteinases, collagenase, or a combination thereof.

In certain embodiments, the contact lens can be sterilized without clattering an architecture or a topography of the coating. In certain non-limiting embodiments, the coated biomaterial can be sterilized using ethylene oxide gas, gamma irradiation, E-beam sterilization, or washing with distilled water.

### 7. EXAMPLES

### 7.1 Example 1: Polyelectrolyte Multilayer Coating for Delivery of IL-4 from Contact Lenses for Eye Disease

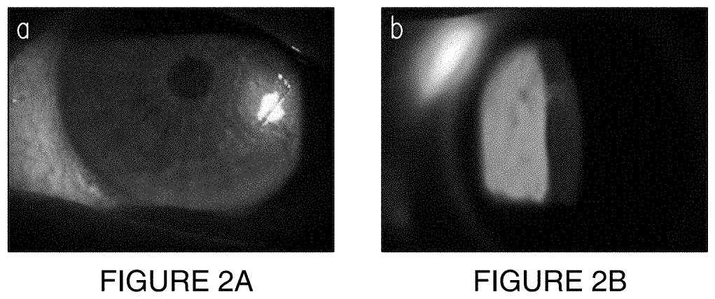

Dry eye disease, which is characterized by aspects such as dryness and irritation of the eye (FIGS. 2A and 2B), is a multifaceted disorder with implications for both tear quality and integrity of the ocular surface. Depending on the population studied and diagnostic criteria used, the prevalence of dry eye disease has been reported to be 7.4 to 33.7% of the population (1), which equates to millions of people currently affected and millions of new cases diagnosed each year in the United States alone. In addition, dry eye syndrome is costly to patients. In 2016, treatments for dry eye (among the markets of the US, France, Germany, Italy, Spain, the UK, Japan, and China) generated sales in excess of 2 billion dollars.

In healthy eyes, the tear film is a multi-component (aqueous, mucin, and lipid components) environment that relies on proper interaction of tear-producing glands, the surface of the eye, and eyelids in order to protect the eye and preserve proper structure and function. When any of the components malfunction, this can cause increased evaporation of the tear film of the eye and/or a decreased production of tears. Both scenarios often result in a surface of the eye that has a higher tear osmolarity than normal, resulting in inflammation and potential damage and irritation to the eyes. Although the initial development of dry eye can be multifactorial in nature, the result is almost always a self-perpetuating cycle of inflammation (1). For example, age is a risk factor for the development of dry eye; though, at first glance, age may not appear to be “immune-mediated.” However, one of the most important tear-producing glands, the lacrimal gland, undergoes changes with aging that includes cell atrophy and fibrosis, leading to diminished tear production, increased osmolarity, and inflammation. In addition, eyelids often suffer from lid laxity during aging, which also can lead to decreased tear production and increased tear evaporation (2).

This inflammatory process is mediated by macrophages; therefore, most, if not all, patients suffering from dry eye could benefit from an immunomodulatory strategy which targets local inflammatory macrophage populations. This approach is different from the current treatments which involve systemic treatments or topical treatments which target non-macrophage cell populations. Eye drops are the mainstay of treatment but come with many drawbacks. Here, an interleukin-4 (IL-4) eluting contact lens for the treatment of dry eye disease is disclosed.

### Materials and Methods

Plasma Treatment, Layer by Layer (LbL) Coating and IL-4 Loading of Biomaterial.

Certain pre-plasma-treated lenses or lenses including an internal wetting agent do not need additional plasma treatment before coating. These types of lenses were rinsed in distilled water to remove residual storage buffer for the dipping procedure, which consists of dipping lenses into oppositely charged solutions of polymers in order to build up layers. Certain contact lenses were cleaned using distilled water and then air-dried prior to irradiation with 60 seconds of argon plasma at 600 W, an argon gas flow of 35 mL/min and a steady-state pressure of 250 mTorr (50 mTorr initial pressure) using an Ion 40 Gas Plasma System (PVA Tepla America, Inc) in order to introduce chemical groups onto the surface that induce a charged surface.

For lenses that need plasma treatment, an adapted radio frequency glow discharge (RFGD) based on a previously developed microwave plasma procedure was used to obtain a negatively charged surface. Maleic anhydride (MA) was used as a monomer for RFGD treatments followed by hydrolysis. Alternating immersion into chitosan and dermatan sulfate solutions (2 mg/mL, 10 minutes each at room temperature) with intermediate washings in water was then performed. This cycle was repeated until the desired number of bilayers was achieved. IL-4 was incubated with dermatan sulfate prior to the coating procedure for IL-4 containing lens groups.

For lenses that necessitate plasma treatment, in particular, maleic anhydride powder (1.5 gr) was placed into a glass plate inside of the machine chamber. Contact lenses were then placed around the plate to a distance of 8.5 cm. After an initial pressure of 50 mTorr was reached, 60 seconds of maleic anhydride plasma treatment was performed at 600 W, an argon gas flow of 35 mL/min and a steady-state pressure of 250 mTorr. Finally, in order to remove the physisorbed maleic anhydride and to hydrolyze the anhydrides and produce carboxylic acid groups (negatively charged at physiological pH), lenses were rinsed for 30 minutes with milli-Q water and then boiled for 20 minutes in fresh milli-Q water.

In order to deposit a conformal coating of nanometric thickness onto the surface of charged lenses, a Layer by Layer (LbL) procedure was performed. Chitosan was chosen as polycation and dermatan sulfate (chondroitin sulfate B) as polyanion. Chitosan was dissolved in 0.5% acetic acid and dermatan sulfate in milli-Q water. Both polyelectrolytes were prepared at a concentration of 2 mg/mL. First, lenses were dipped in chitosan for 10 minutes at room temperature, then lenses were washed 3 times (10, 20 and 30 seconds) in milli-Q water and air dried (pressurized clean air). Next, lenses were dipped in a dermatan sulfate solution for 10 minutes at room temperature. Lenses were washed again in milli-Q water and air-dried. This cycle was repeated until a core coating of 10 bilayers was achieved.

Prior to IL-4 loading onto the lenses, an IL-4 (1.5 μg/mL)-dermatan sulfate (2 mg/mL) mixture was made and incubated overnight at 4° C. in order to complex IL-4 into the polyanion. Then, contact lenses with a 10-bilayer core coating were further coated with 40 bilayers containing IL-4 (PP⁻[CH/DS]₁₀[CH/DS^{IL-4}]ₓ, where x stands for the number of bilayers and DS^{IL-4} stands for dermatan sulfate-bound IL-4). After coating, IL-4 loaded lenses were stored at 4 degrees Celsius. Non-coated lenses were used as controls.

In-Vitro Studies:

Coating characterization. An alcian blue staining was performed to stain the GAG components and reveal the coating. A 1% alcian blue solution was made on 3% acetic acid and adjusted to pH 2.5. Coated lenses and non-coated controls were re-hydrated in distilled water and then immersed into the alcian blue solution for 30 minutes at RT. Then lenses were washed in running tap water for 5 minutes and rinsed 5 minutes in distilled water. Images were taken using a standard optical camera.

IL-4 release assays. Loading efficiency and release assays were performed following manufacturer instructions of R&D Systems IL-4 ELISA kit. First, IL-4 loaded and non-coated (no IL-4) lenses were immersed into 500 μL of a solution 0.05 units/mL chondroitinase ABC and 0.05 units/mL chitosanase in 1×PBS with/without enzymes. Incubation was performed to multiple time points at 37° C., after which 500 μL of the solution was aliquoted and stored at −80° C. until the end of the experiment. After collection, replacement with the fresh solution was performed to continue the release assay. To perform the ELISA assays, 1004 aliquots were used from each sample at each time point.

To determine release profile kinetics; correlation and curve-fitting analyses were performed using the data from cumulative release versus time, until the first time point where the release reaches a plateau, which corresponds to the total release. To corroborate power-law dependence, besides direct curve fitting tests, a linear trend was corroborated using a LOG (cumulative release) versus LOG (time) curve.

### Results and Discussion

IL-4 is known to exert an effect on the inflammatory status of a variety of cell types, with the macrophage being the intended target for this application. Macrophages display a variety of phenotypes based on the external cues and environment with which they reside. Pro-inflammatory or “M1” macrophages are involved in the perpetuation of inflammation, secretion of pro-inflammatory signaling molecules, and destruction of tissues. Anti-inflammatory or “M2” macrophages, on the other hand, are involved in the resolution of inflammation and participate in tissue healing and restorative processes (5). M1 macrophages can be “reprogrammed” to the M2 phenotype when exposed to IL-4 (6).

As M1 macrophages have been shown to be a major mediator in the inflammation associated with dry eye (7), IL-4 administration to the eye will aid in the reprogramming of M1 macrophages to the anti-inflammatory M2 phenotype, which will in turn modify many downstream signaling processes and cellular pathways, ultimately leading to the mitigation of inflammation. To do this, a natural biopolymeric coating was applied to biomaterials that can sustain the release of immune modifying agents (e.g., IL-4) from the surface of a biomaterial over a prolonged period of time (4). This polymeric coating, which consists of a polyelectrolyte multilayer deposition of alternating layers of chitosan and dermatan sulfate complexed to IL-4, can be applied to other medical devices, namely ophthalmic devices. The release of IL-4 from the surface of lens materials can result in a reduction of the inflammatory response in the acute and chronic time points, ultimately leading to the mitigation of symptoms.

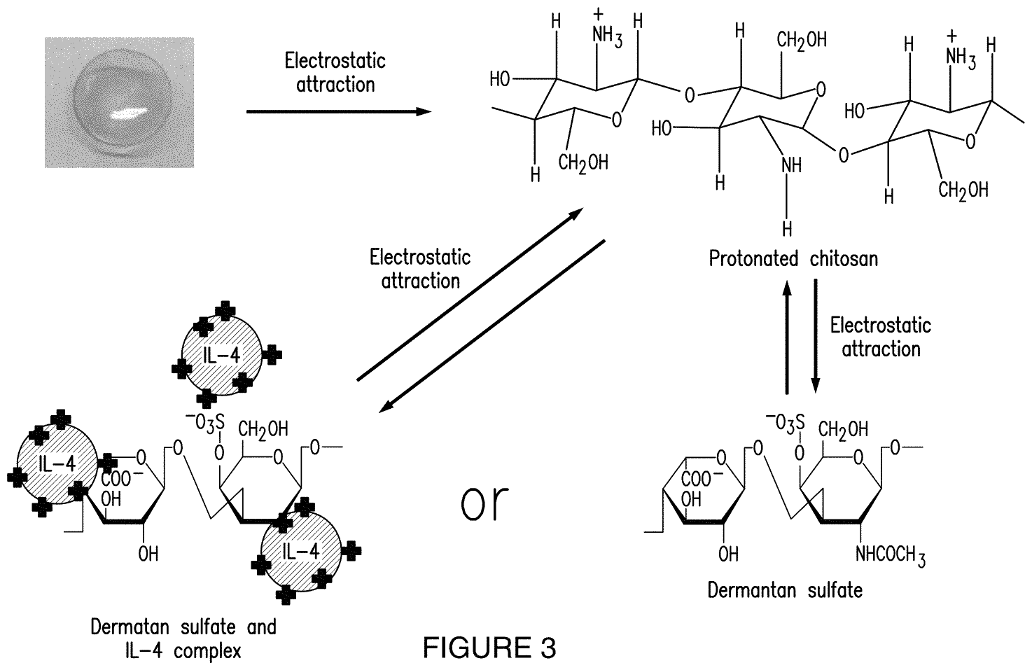

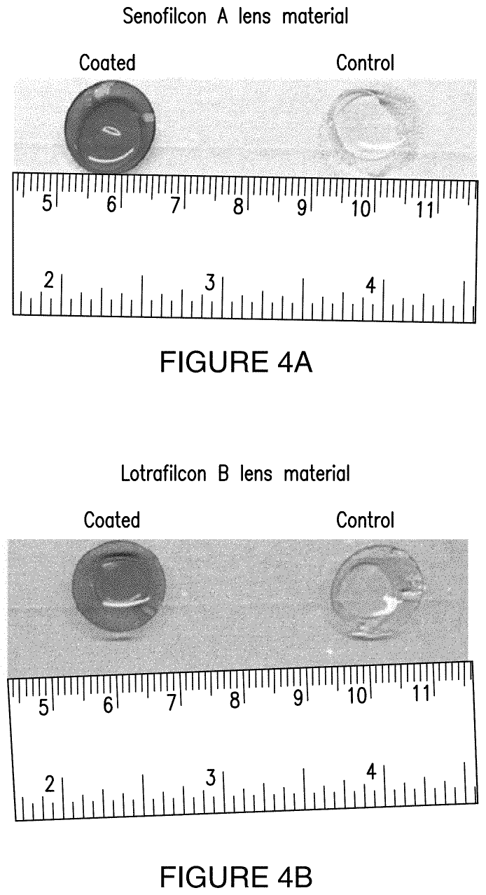

The coating was applied in a uniform way to the surface of a silicone-hydrogel-based contact lens (FIG. 3). In brief, contact lenses were rinsed in distilled water and then dipped in the oppositely charged solutions of polymers in order to build up layers (FIG. 3). Interleukin-4 is able to be complexed into dermatan sulfate and becomes incorporated with the dermatan sulfate-containing layers. To visualize coating adherence, an alcian blue dye stains glycosaminoglycan coating components (FIG. 4) and confirms that the coating can successfully be applied to a lens.

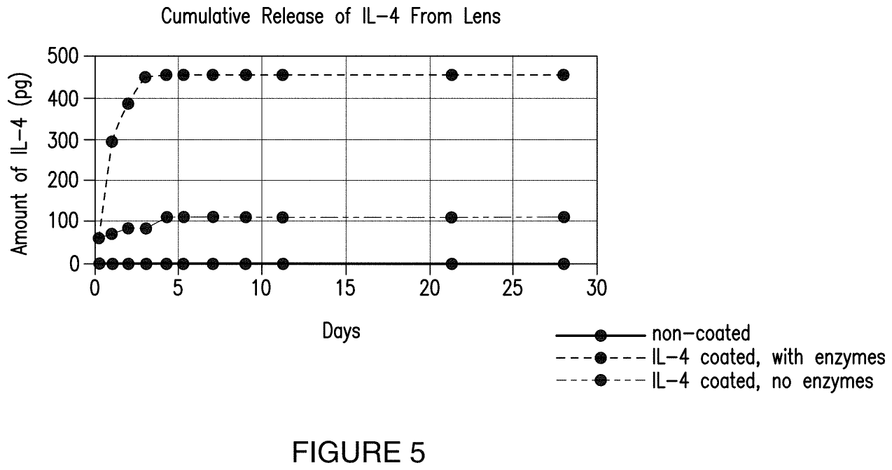

The ability of the lens coating to contain and release IL-4 was assessed next through an in-vitro controlled release experiment in which lenses coated with IL-4-containing-coating were incubated in a solution containing enzymes that mimic those produced by macrophages in-vivo. As opposed to a quick burst release of the drug (as seen with eye drops), the disclosed lens coating is capable of a slower sustained release of the drug over the course of days (FIG. 5). Of note, the release is mainly enzyme driven (as opposed to releasing by diffusion or hydrolysis), as a coated lens incubated in solution void of physiologically relevant enzymes allows the little release of IL-4. To investigate the effects of enzymes on the release of IL-4 from the coating, the coated contact lenses were incubated in a solution for 30 days. As shown in FIG. 5, the coated lens cumulatively released about 100 pg of IL-4 without enzymes. With the treatment of enzymes, the coated lens was able to release about 450 pg of IL-4.

In addition to providing a system to simultaneously modify many aspects of the inflammatory component to dry eye, there are many other potential benefits to the proposed device. A lens capable of releasing IL-4 will provide localized and direct treatment, as opposed to systemic drug administration. A lens capable of extended wear/extended drug delivery will also reduce the need for frequent application of eye drops and/or use of oral treatments, which should boost patient compliance to treatment. Finally, considering that certain topical drugs are oftentimes difficult to acquire in rural or underserved communities, contact lenses are easily accessible to most patients.

The disclosed lens can be placed by the physician. The lens can be placed only once a month or even bi-monthly (reducing the burden of many appointments). If not placed by the physician, contact lens use is exceedingly common, and so this is a technology that is very familiar to many people, which will translate into ease of use. The current gold standard treatments for dry eye are not efficacious with regard to altering disease course or significantly lessening symptoms. A treatment that gives appreciable results (IL-4 can work on treating the underlying cause of the disease rather than treating only the symptoms) will be more motivating for a patient to use, even if this modality proves more challenging than eye drop instillation. If it works well, patients will use it, and physicians will prescribe it.

To date, there are no competitors in the field that are utilizing lens-eluting technology for the targeting of dry eye. Currently, treatment options are limited and focus on either temporary symptomatic relief (e.g., the frequent instillation of artificial tears) or the targeting of limited aspects of the immune system and inflammation (also through the use of eye drops). This includes topical treatment with cyclosporine, of which there are only two FDA approved commercially available formulations (Restasis®, Cequa™) which makes the drug very expensive and sometimes inaccessible to patients. Steroid eye drops are also occasionally used; however, the side effects (e.g., cataract development, glaucoma) can be debilitating. Oral treatments to treat dry eye, such as doxycycline, have their own set of concerns, coupled with the obvious limitation of using a systemic drug to treat a localized problem. With all of these treatments, patient compliance is a major issue, especially when necessitating the need to apply potentially expensive eye drops numerous times a day. Instead of targeting limited aspects of the inflammatory process (as is the case with current treatment regimens), the disclosed systems and methods target a class of cells (macrophages) that are able to respond to an immune modifying agent, interleukin-4 (IL-4), with the direct resolution of the inflammatory response as a result. As opposed to being asymptomatic treatment (as is the instillation of artificial tears), IL-4 is a potent immuno-modulator that can help to correct the underlying condition, with the ultimate goal of changing the course of the disease. Furthermore, most of the active ingredient in prescription eye drops is lost (i.e., most quickly drains from the eye and is not absorbed), resulting in a quick dissipation of a very reduced amount of drug soon after instillation of the eye drop (3). The benefit of the disclosed device is that release will be comparatively slower over a multiple day time course, ultimately resulting in more absorption of drug delivered over a sustained time. The release of IL-4 using such coatings has demonstrated that the release is limited to the areas directly adjacent to the surface of the coated biomaterial, reducing systemic concerns (4).

The disclosed subject matter can reduce the cost of the treatment compared to the cost of prescription eye drop treatments. The most expensive component to the disclosed system, IL-4, has been shown to modify inflammatory processes and alter remodeling processes when eluted from a coating on polypropylene mesh in a mouse model of the healing response and foreign body reaction to synthetic implants (4). The amount eluted in this application was in the nanogram range. Therefore, a very small amount is expected to be required in the current application as well, enabling most of the cost of the device to be limited to the cost of contact lenses and fabrication. Additionally, as this treatment targets the underlying mechanisms of the disease (i.e. dysregulated inflammatory response in macrophages), this treatment can have longer-lasting effects than those which are currently available.

### 7.2 Example 2: Sustained Delivery of IL-4 from Contact Lenses for Macrophage Polarization

### Materials and Methods

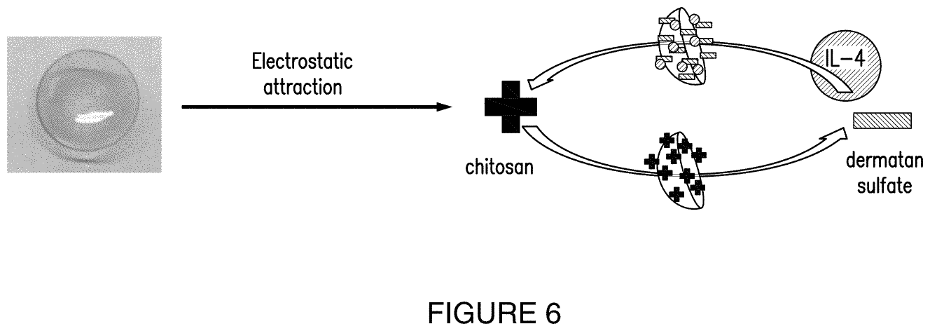

Layer by layer (LbL) coating: A coating containing IL-4 was applied using a layer-by-layer technique where lenses are dipped in oppositely charged polymer solutions to build up layers (FIG. 6). IL-4 is able to be complexed into dermatan sulfate and becomes incorporated with the dermatan sulfate-containing layers.

Coating characterization: To confirm coating adherence, an alcian blue dye was used to stain glycosaminoglycan coating components. The ability of the lens coating to contain and release IL-4 was assessed next through an in-vitro controlled release experiment in which lenses coated with IL-4-containing-coating were incubated in a solution containing enzymes that mimic those produced by ocular macrophages in-vivo.

In-vitro studies: Macrophages were cultured and incubated with either coated/uncoated contact lenses or cytokines known to cause anti-/pro-inflammatory phenotypes. Ability to modify macrophage phenotype was assessed through staining for intracellular arginase (an M2 anti-inflammatory macrophage phenotype marker), through the determination of arginase activity with a biochemical assay, and through the determination of nitric oxide production (an M1 macrophage marker). For advanced imaging characterization, the morphology of soft contact lens, either in their naïve form or coated by our layer-by-layer method with or without incorporated IL-4 was studied by JEOL JSM-6510LV/LGS scanning electron microscopy (SEM). All the lenses were dried by using critical point drying and sputter-coated with gold for 30 seconds with a discharge current of 35 mAmp. The images were taken at 3-5 kV and at ×1000, ×2000, ×7500 magnifications.

### Results and Discussion

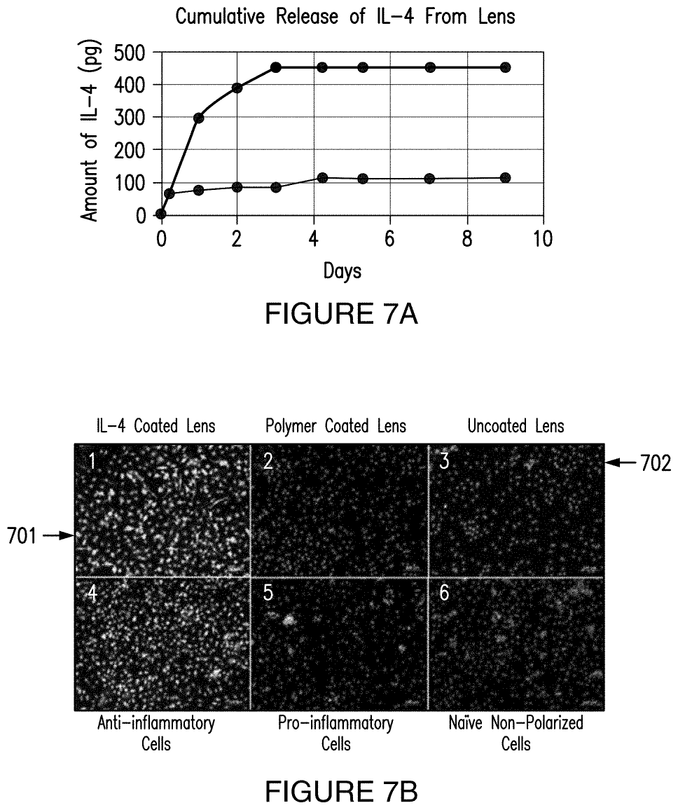

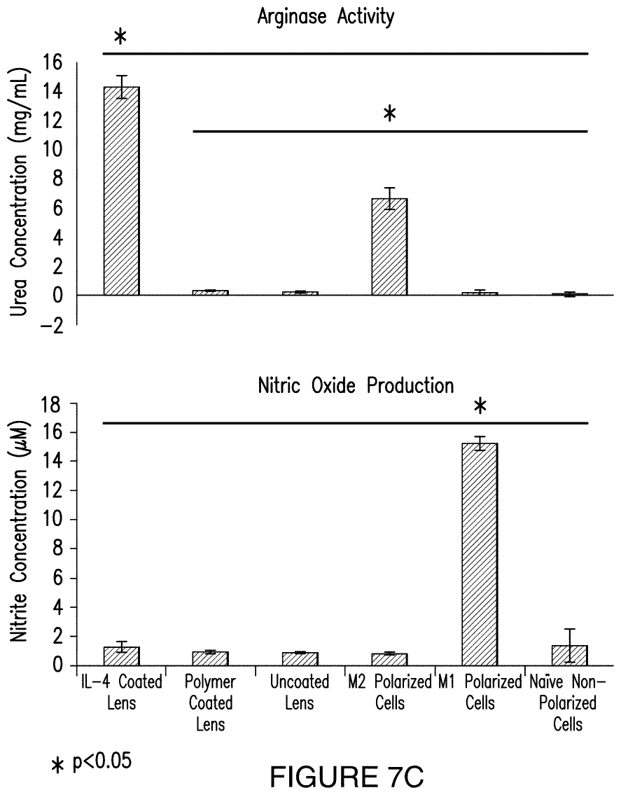

Alcian blue staining confirms that the disclosed biopolymeric coating can successfully be applied in a uniform way to the surface of a silicone-hydrogel-based contact lens (FIG. 4B). In-vitro drug release assays show that, as opposed to a quick burst release of the drug (as seen with eye drops), the disclosed lens coating is capable of a slower sustained release of the drug over the course of days (FIG. 7A, top line). The release is enzyme driven (as opposed to releasing by diffusion or hydrolysis). The coated lens incubated in solution void of physiologically relevant enzymes allows the little release of IL-4 (FIG. 7A-bottom line). As shown in FIG. 7B, non-polarized bone marrow-derived macrophages were incubated with lenses containing IL-4 eluting coating (panel 1), lenses containing non-IL-4 eluting coating (panel 2), lenses with no coating (panel 3), or in media supplemented with 20 ng/mL IL-4 (panel 4), media supplemented with 20 ng/mL INF-γ and 100 ng/mL lipopolysaccharide (LPS) (panel 5), or in media with no supplementation (panel 6). Cells in panel 4 serve as a positive control for producing anti-inflammatory M2 macrophages and cells in panel 5 serve as a positive control for producing pro-inflammatory M1 macrophages. Cell populations are deemed more anti-inflammatory if intracellular immunofluorescent arginase staining is more intense, as increased arginase production is a hallmark of M2 anti-inflammatory macrophages. For these in-vitro culture experiments, stainings for intracellular arginase 701 and nucleus 702 are shown in FIG. 7B, as well as assessment of arginase activity with a biochemical assay (FIG. 7C, top panel), shows that the IL-4 released from the disclosed lens device is capable of programming target cells to an anti-inflammatory phenotype that can mitigate dry eye symptoms. The IL-4 coated lenses did not cause any appreciable production of nitric oxide (FIG. 7C, bottom panel), which is a marker of the pro-inflammatory M1 macrophage phenotype that is responsible for the perpetuation of dry eye disease. The enhanced arginase immunofluorescent staining in IL-4 coated lens groups as compared to the positive control group for M2 macrophages (FIG. 7B, panels 1 and 4) and enhanced arginase activity (in IL-4 coated lens groups) as compared to the positive control group for M2 macrophages (FIG. 7C, top panel, groups 1 and 4) show that the efficacy and potency of IL-4 is increased when complexed with dermatan sulfate and released from the disclosed coating.

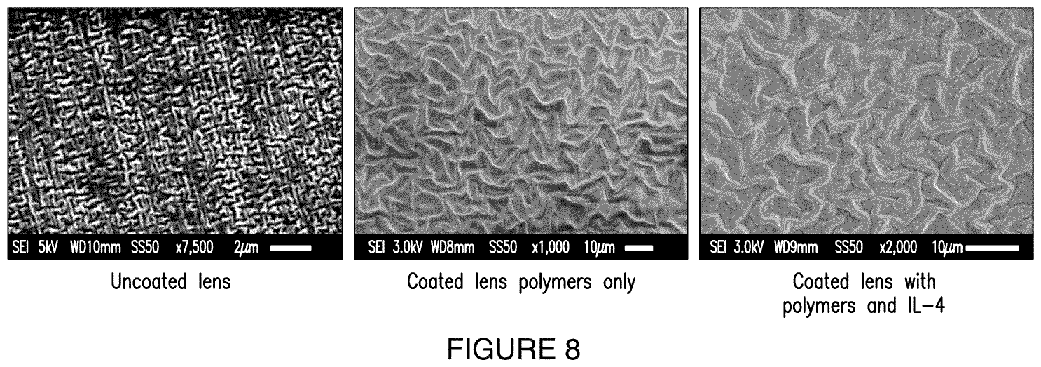

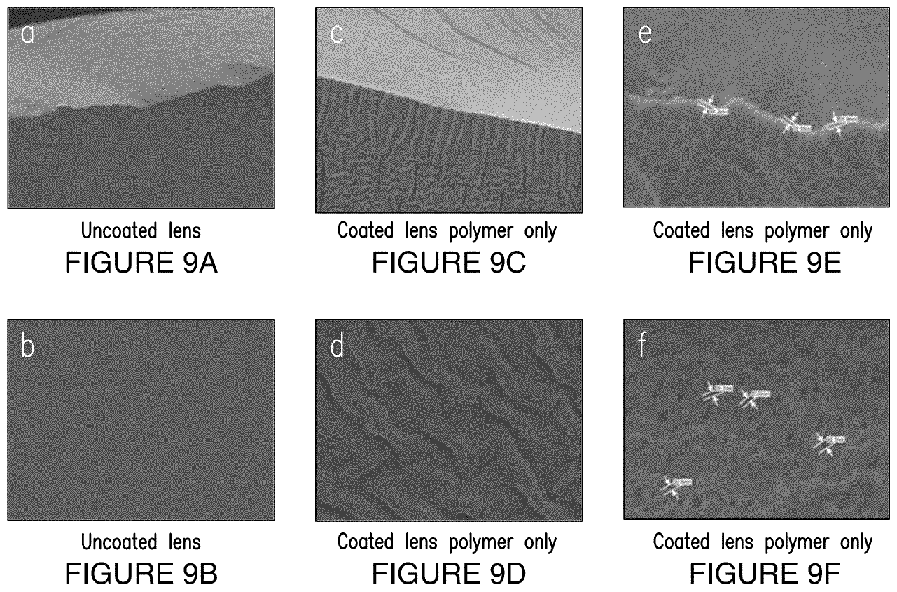

For critically dried samples using the critical point drying, scanning electron microscopy imaging (FIG. 8) of the coated lenses shows, regardless of incorporated IL-4, the obvious presence of coatings with similar morphology of lenses. The naïve uncoated lenses were morphologically very distinct, however. To measure the thickness and further characterize surface features of the coating, samples were instead prepared with cryo-fracture technique (FIG. 3). A cross-sectional view (FIG. 9A) and a frontal view (FIG. 9B) of uncoated lenses show distinct morphology consistent with a naïve untreated contact lens. Lenses coated with polymers (FIGS. 9C-9F) show features corroborating the presence of an overlying coating. A cross-sectional view (FIG. 9C) and a frontal view (FIG. 9D) of polymer-coated lenses show convoluted undulating features of the polymers. Higher magnification of the cross-section view (FIG. 9E) shows a conformal coating of thickness ranging from 52 nm to 56.3 nm. The high magnification frontal view (FIG. 9F) shows the presence of pores within the polymer coating with diameters ranging from 25.2 nm to 42.3 nm.

IL-4 containing polymeric coatings can be applied successfully to silicone-hydrogel based contact lenses and provides for an efficient and effective delivery vehicle that allows for a sustained and local release of optimal concentrations of a therapeutic drug, while minimizing product loss. Targeting the macrophage-centric pathway of dry eye inflammation can lead to a longer-term, more curative treatment outcome, leading to prolonged symptom relief with an infrequent dosing regimen.

### 9. REFERENCES

J. L. Gayton, Etiology, prevalence, and treatment of dry eye disease, Clinical Ophthalmology 3 (2009) 405-12.
C. S. de Paiva. Effects of Aging in Dry Eye, International ophthalmology clinics. 57(2) (2017) 47-64.
A. Farkouh, P. Frigo, M. Czejka, Systemic side effects of eye drops: a pharmacokinetic perspective, Clinical ophthalmology 10 (2016) 2433-41.
D. Hachim, S. T. LoPresti, C. C. Yates, B. N. Brown, Shifts in macrophage phenotype at the biomaterial interface via IL-4 eluting coatings are associated with improved implant integration, Biomaterials 112 (2017) 95-107.
Liu Y C, Zou X B, Chai Y F, & Yao Y M (2014) Macrophage polarization in inflammatory diseases. Int J Biol Sci 10(5):520-529.
I. G. Luzina, A. D. Keegan, N. M. Heller, G. Rook, T. Shea-Donohue, S. P. Atamas, Regulation of inflammation by interleukin-4: a review of “alternatives”, Journal of Leukocyte Biology 92 (4) (2012) 753-64.
I. C. You, T. G. Coursey, F. Bian, F. L. Barbosa, C. S. de Paiva, S. C. Pflugfelder, Macrophage Phenotype in the Ocular Surface of Experimental Murine Dry Eye Disease, Archivum immunologiae et therapiae experimentalis, 63 (4) (2015) 299-304.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

Various patents and patent applications are cited herein, the contents of which are hereby incorporated by reference herein in their entireties.

What is claimed is:

1. A contact lens for treating an ocular disorder comprising:
(a) a lens body; and
(b) a uniform coating thereon,
wherein the coating comprises a plurality of polycation layers and a plurality of polyanion layers, and wherein at least one layer of the coating comprises an M2 polarizing active agent.

2. The contact lens of claim 1, wherein the lens body is a silicone hydrogel lens body.

3. The contact lens of claim 1, wherein the lens body is selected from the group consisting of balafilcon A, lotrafilcon A, lotrafilcon B, etafilcon A, narafilcon A, galyfilcon A, senofilcon A, ocufilcon D, hioxifilicon A, enfilcon A, comfilcon A, nesofilcon A, filicon II 3, deleficon A, methafilcon A, methafilcon B, vifilcon A, phemfilcon A, nelfilcon A, stenfilcon A, polymacon, hefilcon B, tetrafilcon A, omafilcon A, polymacon B, hilafilcon B, alphafilcon A, and combinations thereof.

4. The contact lens of claim 1, wherein the ocular disorder is selected from the group consisting of allergic, bacterial, chemical or viral conjunctivitis, blepharitis, dry eye syndrome, sub-conjunctival hematomas, corneal abrasion, uveitis, and combinations thereof.

5. The contact lens of claim 1, wherein the polycation in at least one polycation layer is selected from the group consisting of a polysaccharide, a protein, a synthetic polypeptide, a synthetic polyamine, a synthetic polymer, a positively charged polymer or copolymer, and combinations thereof.

6. The contact lens of claim 1, wherein the polyanion in at least one polyanion layer is selected from the group consisting of a polysaccharide, a protein, a synthetic polypeptide, a synthetic polyamine, a synthetic polymer, and combinations thereof.

7. The contact lens of claim 1, wherein the M2 polarizing active agent is selected from the group consisting of IL-4, IL-10, IL-13, TGF-β, HGF, and combinations thereof.

8. The contact lens of claim 1, wherein a thickness of the coating is from about 0.5 nm to about 500 μm.

9. The contact lens of claim 1, wherein the at least one layer of the coating comprises dermatan sulfate and the M2 polarizing active agent, and the M2 polarizing active agent and the dermatan sulfate are present in a ratio between about 1:10 to about 1:2000.

10. The contact lens of claim 1, wherein the coating comprises a macrophage-related enzyme or protein that adjusts a release rate of the active agent from the coating.

11. The contact lens of claim 1, wherein the coating is placed on the lens body without altering an optical property of the contact lens, wherein the optical property includes vision correction.

12. The contact lens of claim 1, wherein the coating is uniformly coated on a surface of the lens body without being exposed to plasma gas.

13. A method for treating ocular disorders comprising:
placing a contact lens on a surface of an eye, wherein the contact lens comprises
(a) a lens body; and
(b) a uniform coating thereon,
wherein the coating comprises a plurality of polycation layers and a plurality of polyanion layers, and wherein at least one layer of the coating comprises an M2 polarizing active agent.

14. The method of claim 13, further comprising alleviating at least one symptom of the ocular disorder, wherein the at least one symptom is selected from the group consisting of redness, itching, burning, foreign body sensation, watery eyes, dry eyes, swelling, pain, clouding of vision, secretion of pus, sticking eyelids, altered sensitivity to light, and a combination of thereof.

15. The method of claim 13, further comprising sterilizing the contact lens without altering an architecture or a topography of the coating.

16. The method of claim 13, wherein the contact lens is worn continuously for about 30 days.

17. The method of claim 13, further comprising delivering a supplement solution to the eye.

18. The method of claim 17, wherein the supplement solution comprises artificial tears, a macrophage-related enzyme, or a combination thereof.

19. The method of claim 13, wherein the coating is degraded without altering an optical property of the contact lens, wherein the optical property includes vision correction.

20. The method of claim 13, wherein the contact lens simultaneously corrects vision of a subject during the release of active agents from the coating.
